(19) United States
(12) Patent Application Publication
(10) Pub. No.: US 2018/0155743 A1
(43) Pub. Date: Jun. 7, 2018

(54) PRODUCTION OF PROPANOL AND/OR PROPIONIC ACID

(71) Applicant: EVONIK DEGUSSA GMBH, Essen (DE)

(72) Inventors: Thomas HAAS, Münster (DE); Simon BECK, Münster (DE); Martin DEMLER, Dorsten (DE); Thomas BÜLTER, Duisburg (DE)

(73) Assignee: EVONIK DEGUSSA GMBH, Essen (DE)

(21) Appl. No.: 15/574,365

(22) PCT Filed: Jul. 28, 2016
(86) PCT No.: PCT/EP2016/068036

§ 371 Date: Nov. 15, 2017

(30) Foreign Application Priority Data
Aug. 12, 2015 (EP) 15180693.2

(51) Int. Cl. C12P 7/04; C12P 7/52

(52) U.S. Cl. CPC C12P 7/04; C12P 7/52

(57) ABSTRACT

The present invention relates to a method of producing propanol and/or propionic acid from a carbon source in aerobic conditions, the method comprising:

(a) step of producing ethanol and/or acetate from the carbon source in aerobic conditions, comprising
(i) contacting a reaction mixture comprising
a first acetogenic microorganism in an exponential growth phase;
free oxygen; and
a second acetogenic microorganism in a stationary phase
   wherein the first and second acetogenic microorganism is capable of converting the carbon source to the acetate and/or ethanol; and
   (b) step of contacting the acetate and/or ethanol from step (a) with a third microorganism capable of converting the acetate and/or ethanol to propanol and/or propionic acid.

### FIELD OF THE INVENTION

The present invention relates to a biotechnological method for producing propanol and/or propionic acid. In particular, the method may use carbon monoxide and/or carbon dioxide as the starting material and may be carried out in aerobic conditions.

### BACKGROUND OF THE INVENTION

Propanol is a solvent used in the pharmaceutical industry for resins and cellulose esters amongst other compounds. This solvent, which is better known as isopropanol or isopropyl alcohol, is widely used on printing ink and in the printing industry. 1-propanol is produced in nature by the decomposition of organic materials by a variety of microorganisms and may be found in plants and fusel oil. 1-propanol can also be produced from petrochemically-derived ethene by a reaction with carbon monoxide and hydrogen to give propionaldehyde, which is then hydrogenated. It is also a byproduct of methanol manufacture and may be produced from propane directly or from acrolein.

Propanol has other potential uses. One of the important uses of propanol is that it can be readily dehydrated to produce propylene which is one of the largest chemical commodities in the world. For example, isopropanol (IPA) is currently produced using propylene. In particular, by one of two processes that use petrochemically derived precursors: (1) a two-step (indirect) process during which propylene is hydrogenated and then hydrolysed using acid and water or (2) a one-step (direct) process during which propylene is hydrogenated using an acid catalyst. IPA is one of the more important solvents used in the chemical industry. It is also an important chemical intermediate. It is a component of cleaners, disinfectants, room sprays, lacquers and thinners, adhesives, pharmaceuticals, cosmetics and toiletries. It is also used as an extractant and as a dehydrating agent. IPA is also used as a gasoline additive, to dissolve water and ice in fuel lines and tanks thereby preventing the water from accumulating in the fuel lines and freezing at low temperatures.

The global demand for isopropanol and propylene continues to increase at a rate of about 3% per year. Since the current methods of producing isopropanol and propylene are primarily manufactured from petroleum, the costs of these compounds will be based on the costs of petroleum. These compounds are also obtained by cracking gasoline or petroleum which is bad for the environment. Accordingly, there is a need for an environmentally friendly and bio-based alternative to the petro-based production process is the production of IPA by fermentation from renewable biomass. However, to be viable and outperform in the current petrochemical IPA market, a fermentative process for the production of IPA must be cost-effective.

### DESCRIPTION OF THE INVENTION

The present invention provides a biotechnological means of producing propanol and/or propionic acid from a carbon source in aerobic conditions. The carbon source may comprise carbon dioxide and/or carbon monoxide. In particular, the method comprises at least two parts. One part that involves the formation of acetate and/or ethanol from a carbon source and a further part which involves the use of the acetate and/or ethanol in the formation of propanol and/or propionic acid.

In one aspect of the present invention, there is provided a method of producing propanol and/or propionic acid from a carbon source in aerobic conditions, the method comprising:
(a) step of producing ethanol and/or acetate from the carbon source in aerobic conditions, comprising
   (i) contacting a reaction mixture comprising
      a first acetogenic microorganism in an exponential growth phase;
      free oxygen; and
      a second acetogenic microorganism in a stationary phase
wherein the first and second acetogenic microorganism is capable of converting the carbon source to the acetate and/or ethanol; and
(b) step of contacting the acetate and/or ethanol from step (a) with a third microorganism capable of converting the acetate and/or ethanol to propanol and/or propionic acid.

A microorganism capable of converting acetate and/or ethanol to propanol and/or propionic acid may refer to any microorganism that may be able to carry out fermentative production of propanol and/or propionic acid. This microorganism may be a propionogen. Propionogens are C3-producing microorganisms. In particular, propionogens refers to any microorganism which may be capable of converting syngas intermediates, such as ethanol and acetate, to propionic acid and propanol. The terms “propionogen” or “C3-producing microorganism” refers to microorganisms which, when contacted with a substrate, convert the substrate to propanol and/or propionic acid. These microorganisms may produce the appropriate enzymes intracellularty and/or extracellularty. These propanol and/or propionic acid producing microorganisms may be capable of utilising starting material for propanol and/or propionic acid fermentation that may be waste materials. For instance, syngas and the ethanol and/or acetate derived from syngas may be utilized for the propanol and/or propionic acid production. This is particularly advantageous as inexpensive starting materials can be utilized that would originally have been considered waste. This also enables the removal of waste which consequently reduces environmental pollution. In one example, the propionogen according to any aspect of the present invention may use at least the methylmalonyl-succinate pathway (FIG. 1) or the lactate-acrylate pathway (FIG. 2) to produce propionate from acetate and/or alcohol.

In particular, the propionogen used according to any aspect of the present invention may be selected from the group consisting of Clostridium neopropionicum, Clostridium propionicum, Pelobacter propionicus, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii.

In one example, the third microorganism may be any eukaryotic or prokaryotic microorganism that may be genetically modified. More in particular, the third microorganism may be a strain selected from the group consisting of Escherichia sp., Etwinia sp., Serratia sp., Providencia sp., Corynebacteria sp., Pseudomonas sp., Leptospira sp., Salmonellar sp., Brevibacteria sp., Hypomononas sp., Chromobacterium sp., Norcardia sp., fungi and yeasts. Even more in particular, the third microorganism may be selected from Escherichia sp. For example, the third microorganism according to any aspect of the present invention may be Escherichia coli. The third microorganism may be a genetically modified organism comprising increased expression relative to the wild type cell of propionate CoA-transferase (AJ276553) (E₁), lactoyl-CoA dehydratase (JN244651-3) (E₂) and acryloyl-CoA reductase (JN244654-6) (E₃). Kandasamy V. (2013) discloses a method of producing a genetic organism as such. Kandasamy V. also discloses a means of measuring the expression of enzymes E₁, E₂ and E₃ to determine if any one of these enzymes have increased expression relative to the wild type cell.

The term “acetate” as used herein, refers to both acetic acid and salts thereof, which results inevitably, because as known in the art, since the microorganisms work in an aqueous environment, and there is always a balance between salt and acid present.

In particular, the second acetogenic microorganism in a post exponential phase may be in the stationary phase of the cell. The acetogenic cells in the log phase allow for any other acetogenic cells in the aqueous medium to produce acetate and/or ethanol in the presence of oxygen. The concentration of acetogenic cells in the log phase may be maintained in the reaction mixture. Therefore, at any point in time in the reaction, the reaction mixture comprises acetogenic cells in the log phase and acetogenic cells in another growth phase, for example in the stationary phase.

A skilled person would understand the different growth phases of microorganisms and the methods to measure them and identify them. In particular, most microorganisms in batch culture, may be found in at least four different growth phases; namely they are: lag phase (A), log phase or exponential phase (B), stationary phase (C), and death phase (D). The log phase may be further divided into the early log phase and mid to late log/exponential phase. The stationary phase may also be further distinguished into the early stationary phase and the stationary phase. For example, Cotter, J. L., 2009, Najafpour. G., 2006, Younesi, H., 2005, and Köpke, M., 2009 disclose different growth phases of acetogenic bacteria. In particular, the growth phase of cells may be measured using methods taught at least in Shuler M L, 1992 and Fuchs G., 2007.

The lag phase is the phase immediately after inoculation of the cells into a fresh medium, the population remains temporarily unchanged. Although there is no apparent cell division occurring, the cells may be growing in volume or mass, synthesizing enzymes, proteins, RNA, etc., and increasing in metabolic activity. The length of the lag phase may be dependent on a wide variety of factors including the size of the inoculum; time necessary to recover from physical damage or shock in the transfer; time required for synthesis of essential coenzymes or division factors; and time required for synthesis of new (inducible) enzymes that are necessary to metabolize the substrates present in the medium.

The exponential (log) phase of growth is a pattern of balanced growth wherein all the cells are dividing regularly by binary fission, and are growing by geometric progression. The cells divide at a constant rate depending upon the composition of the growth medium and the conditions of incubation. The rate of exponential growth of a bacterial culture is expressed as generation time, also the doubling time of the bacterial population. Generation time (G) is defined as the time (t) per generation (n=number of generations). Hence, G=t/n is the equation from which calculations of generation time derive. The exponential phase may be divided into the (i) early log phase and (ii) mid to late log/exponential phase. A skilled person may easily identify when a microorganism, particularly an acetogenic bacteria, enters the log phase. For example, the method of calculating the growth rate of acetogenic bacteria to determine if they are in the log phase may be done using the method taught at least in Henstra A. M., 2007. In particular, the microorganism in the exponential growth phase according to any aspect of the present invention may include cells in the early log phase and mid to late log/exponential phase.

The stationary phase is the phase where exponential growth ends as exponential growth cannot be continued forever in a batch culture (e.g. a closed system such as a test tube or flask). Population growth is limited by one of three factors: 1. exhaustion of available nutrients; 2. accumulation of inhibitory metabolites or end products; 3. exhaustion of space, in this case called a lack of “biological space”. During the stationary phase, if viable cells are being counted, it cannot be determined whether some cells are dying and an equal number of cells are dividing, or the population of cells has simply stopped growing and dividing. The stationary phase, like the lag phase, is not necessarily a period of quiescence. Bacteria that produce secondary metabolites, such as antibiotics, do so during the stationary phase of the growth cycle (Secondary metabolites are defined as metabolites produced after the active stage of growth).

The death phase follows the stationary phase. During the death phase, the number of viable cells decreases geometrically (exponentially), essentially the reverse of growth during the log phase. In one example, where O₂ is present in the reaction mixture according to any aspect of the present invention, the first acetogenic bacteria may be in an exponential growth phase and the other acetogenic bacteria may be in any other growth phase in the lifecycle of an acetogenic microorganism. In particular, according to any aspect of the present invention, the acetogenic bacteria in the reaction mixture may comprise one acetogenic bacteria in an exponential growth phase and another in the stationary phase. In the presence of oxygen, without the presence of the acetogenic bacteria in an exponential growth, the acetogenic bacteria in the stationary phase may not be capable of producing acetate and/or ethanol. This phenomenon is confirmed at least by Brioukhanov, 2006, Imlay, 2006, Lan, 2013 and the like. The inventors thus surprisingly found that in the presence of acetogenic bacteria in an exponential growth, the acetogenic bacteria in any growth phase may aerobically respire and produce acetate and/or ethanol at more than or equal to the amounts produced when the reaction mixture was absent of oxygen. In one example, the acetogenic bacteria in the exponential growth phase may be capable of removing the free oxygen from the reaction mixture, providing a suitable environment (with no free oxygen) for the acetogenic bacteria in any growth phase to metabolise the carbon substrate to produce acetate and/or ethanol.

In another example, the aqueous medium may already comprise acetogenic bacteria in any growth phase, particularly in the stationary phase, in the presence of a carbon source. In this example, there may be oxygen present in the carbon source supplied to the aqueous medium or in the aqueous medium itself. In the presence of oxygen, the acetogenic bacteria may be inactive and not produce acetate and/or ethanol prior to the addition of the acetogenic bacteria in the exponential growth phase. In this very example, the acetogenic bacteria in the exponential growth phase may be added to the aqueous medium. The inactive acetogenic bacteria already found in the aqueous medium may then be activated and may start producing acetate and/or ethanol.

In a further example, the acetogenic bacteria in any growth phase may be first mixed with the acetogenic bacteria in the exponential growth phase and then the carbon source and/or oxygen added.

According to any aspect of the present invention, a microorganism in the exponential growth phase grown in the presence of oxygen may result in the microorganism gaining an adaptation to grow and metabolise in the presence of oxygen. In particular, the microorganism may be capable of removing the oxygen from the environment surrounding the microorganism. This newly acquired adaptation allows for the acetogenic bacteria in the exponential growth phase to rid the environment of oxygen and therefore produce acetate and ethanol from the carbon source. In particular, the acetogenic bacteria with the newly acquired adaptation allows for the bacteria to convert the carbon source to acetate and/or ethanol.

In one example, the acetogenic bacteria in the reaction mixture according to any aspect of the present impression may comprise a combination of cells: cells in the log phase and cells in the stationary phase. In the method according to any aspect of the present invention the acetogenic cells in the log phase may comprise a growing rate selected from the group consisting of 0.01 to 2 h⁻¹, 0.01 to 1 h⁻¹, 0.05 to 1 h⁻¹, 0.05 to 2 h⁻¹ 0.05 to 0.5 h⁻¹ and the like. In one example, the OD₆₀₀ of the cells of the log phase acetogenic cells in the reaction mixture may be selected from the range consisting of 0.001 to 2, 0.01 to 2, 0.1 to 1, 0.1 to 0.5 and the like. A skilled person would be able to use any method known in the art to measure the OD₆₀₀ and determine the growth rate of the cells in the reaction mixture and/or to be added in the reaction mixture. For example, Koch (1994) may be used. In particular, bacterial growth can be determined and monitored using different methods. One of the most common is a turbidity measurement, which relies upon the optical density (OD) of bacteria in suspension and uses a spectrophotometer. The OD may be measured at 600 nm using a UV spectrometer.

In order to maintain the concentration of the first and second acetogenic bacteria in the reaction mixture, a skilled person may be capable of extracting a sample at fixed time points to measure the OD₆₀₀, pH, concentration of oxygen and concentration of ethanol and/or higher alcohols formed. The skilled person would then be able to add the necessary component(s) to maintain the concentration of first and second acetogenic bacteria in the reaction mixture and to ensure an optimum environment is maintained for the production of ethanol and/or acetate.

The term “acetogenic bacteria” as used herein refers to a microorganism which is able to perform the Wood-Ljungdahl pathway and thus is able to convert CO, CO₂ and/or hydrogen to acetate. These microorganisms include microorganisms which in their wild-type form do not have a Wood-Ljungdahl pathway, but have acquired this trait as a result of genetic modification. Such microorganisms include but are not limited to E. coli cells. These microorganisms may be also known as carboxydotrophic bacteria. Currently, 21 different genera of the acetogenic bacteria are known in the art (Drake et al., 2006), and these may also include some clostridia (Drake & Kusel, 2005). These bacteria are able to use carbon dioxide or carbon monoxide as a carbon source with hydrogen as an energy source (Wood, 1991). Further, alcohols, aldehydes, carboxylic acids as well as numerous hexoses may also be used as a carbon source (Drake et al., 2004). The reductive pathway that leads to the formation of acetate is referred to as acetyl-CoA or Wood-Ljungdahl pathway.

In particular, the acetogenic bacteria may be selected from the group consisting of Acetoanaerobium notera (ATCC 35199), Acetonema longum (DSM 6540), Acetobacterium carbinolicum (DSM 2925), Acetobacterium malicum (DSM 4132), Acetobacternum species no. 446 (Morinaga et al., 1990, J. Biotechnol., Vol. 14, p. 187-194), Acetobacterium wieringae (DSM 1911), Acetobacternum woodii (DSM 1030), Alkalibaculum bacchi (DSM 22112), Archaeoglobus fulgidus (DSM 4304), Blautia producta (DSM 2950, formerly Ruminococcus productus, formerly Peptostreptococcus productus), Butyribacterium methylotrophicum (DSM 3468), Clostridium aceticum (DSM 1496), Clostridium autoethanogenum (DSM 10061, DSM 19630 and DSM 23693), Clostridium carboxidivorans (DSM 15243), Clostridium coskatii (ATCC no. PTA-10522), Clostridium drakei (ATCC BA-623), Clostridium formicoaceticum (DSM 92), Clostridium glycolicum (DSM 1288), Clostridium jungdahlii (DSM 13528), Clostridium Ijungdahlii C-01 (ATCC 55988), Clostridium Ijungdahlii ERI-2 (ATCC 55380), Clostridium Ijungdahlii O-52 (ATCC 55989), Clostridium mayombei (DSM 6539), Clostridium methoxybenzovorans (DSM 12182), Clostridium ragsdalei (DSM 15248), Clostridium scatologenes (DSM 757), Clostridium species ATCC 29797 (Schmidt et al., 1986, Chem. Eng. Commun., Vol. 45, p. 61-73), Desulfotomaculum kuznetsovii (DSM 6115), Desulfotomaculum thermobezoicum subsp. thermosyntrophicum (DSM 14055), Eubacterium limosum (DSM 20543), Methanosarcina acetivorans C2A (DSM 2834), Moorella sp. HUC22-1 (Sakai et al., 2004), Moorella thermoacetica (DSM 521, formerly Clostridium thermoaceticum), Moorella thermoautotrophica (DSM 1974), Oxobacter pfennigii (DSM 322), Sporomusa aerivorans (DSM 13326), Sporomusa ovata (DSM 2662), Sporomusa silvacetica (DSM 10669), Sporomusa sphaeroides (DSM 2875), Sporomusa termitida (DSM 4440) and Thermoanaerobacter kivui (DSM 2030, formerly Acetogenium kivui). More in particular, the strain ATCC BAA-624 of Clostridium carboxidivorans may be used. Even more in particular, the bacterial strain labelled “P7” and “P11” of Clostridium carboxidivorans as described for example in U.S. 2007/0275447 and U.S. 2008/0057554 may be used.

Another particularly suitable bacterium may be Clostridium Ijungdahlii. In particular, strains selected from the group consisting of Clostridium Ijungdahlii PETC, Clostridium Ijungdahlii ERI2, Clostridium Ijungdahlii COL and Clostridium Ijungdahlii O-52 may be used in the conversion of synthesis gas to hexanoic acid. These strains for example are described in WO 98/00558, WO 00/68407, ATCC 49587, ATCC 55988 and ATCC 55989. The first and second acetogenic bacteria used according to any aspect of the present invention may be the same or different bacteria. For example, in one reaction mixture the first acetogenic bacteria may be Clostridium Ijungdahlii in the log phase and the second acetogenic bacteria may be Clostridium Ijungdahlii in the stationary phase. In another example, in the reaction mixture the first acetogenic bacteria may be Clostridium Ijungdahlii in the log phase and the second acetogenic bacteria may be Clostridium carboxidivorans in the stationary phase.

In the reaction mixture according to any aspect of the present invention, there may be oxygen present (i.e. aerobic conditions are used). It is advantageous to incorporate O₂ in the reaction mixture and/or gas flow being supplied to the reaction mixture as most waste gases including synthesis gas comprises oxygen in small or large amounts. It is difficult and costly to remove this oxygen prior to using synthesis gas as a carbon source for production of higher alcohols. The method according to any aspect of the present invention allows the production of at least one higher alcohol without the need to first remove any trace of oxygen from the carbon source. This allows for time and money to be saved.

More in particular, the O₂ concentration in the gas flow may be may be present at less than 1% by volume of the total amount of gas in the gas flow. In particular, the oxygen may be present at a concentration range of 0.000005 to 2% by volume, at a range of 0.00005 to 2% by volume, 0.0005 to 2% by volume, 0.005 to 2% by volume, 0.05 to 2% by volume, 0.00005 to 1.5% by volume, 0.0005 to 1.5% by volume, 0.005 to 1.5% by volume, 0.05 to 1.5% by volume, 0.5 to 1.5% by volume, 0.00005 to 1% by volume, 0.0005 to 1% by volume, 0.005 to 1% by volume, 0.05 to 1% by volume, 0.5 to 1% by volume, 0.55 to 1% by volume, 0.60 to 1% by volume, particularly at a range of 0.60 to 1.5%, 0.65 to 1%, and 0.70 to 1% by volume in the gas phase of the gas flow and/or in the medium. In particular, the acetogenic microorganism is particularly suitable when the proportion of O₂ in the gas phase/flow is about 0.00005, 0.0005, 0.005, 0.05, 0.5, 0.6, 0.7, 0.8, 0.9, 1, 1.5, 2% by volume in relation to the volume of the gas in the gas flow. A skilled person would be able to use any one of the methods known in the art to measure the volume concentration of oxygen in the gas flow. In particular, the volume of oxygen may be measured using any method known in the art. In one example, a gas phase concentration of oxygen may be measured by a trace oxygen dipping probe from PreSens Precision Sensing GmbH. Oxygen concentration may be measured by fluorescence quenching, where the degree of quenching correlates to the partial pressure of oxygen in the gas phase. Even more in particular, the first and second microorganisms according to any aspect of the present invention are capable of working optimally in the aqueous medium when the oxygen is supplied by a gas flow with concentration of oxygen of less than 1% by volume of the total gas, in about 0.015% by volume of the total volume of gas in the gas flow supplied to the reaction mixture.

According to any aspect of the present invention, the aerobic conditions in which the carbon source is converted to ethanol and/or acetate in the reaction mixture refers to gas surrounding the reaction mixture. The gas may comprise at least 1% by volume of the total gas of oxygen and other gases including carbon sources such as CO, CO₂ and the like.

The aqueous medium according to any aspect of the present invention may comprise oxygen. The oxygen may be dissolved in the medium by any means known in the art. In particular, the oxygen may be present at 0.5 mg/L in the absence of cells. In particular, the dissolved concentration of free oxygen in the aqueous medium may at least be 0.01 mg/L. In another example, the dissolved oxygen may be about 0.01, 0.02, 0.03, 0.04, 0.05, 0.1, 0.2, 0.3, 0.4, 0.5 mg/L. In particular, the dissolved oxygen concentration may be 0.01-0.5 mg/L, 0.01-0.4 mg/L, 0.01-0.3 mg/L, 0.01-0.1 mg/L. In particular, the oxygen may be provided to the aqueous medium in a continuous gas flow. More in particular, the aqueous medium may comprise oxygen and a carbon source comprising CO and/or CO₂. More in particular, the oxygen and a carbon source comprising CO and/or CO₂ is provided to the aqueous medium in a continuous gas flow. Even more in particular, the continuous gas flow comprises synthesis gas and oxygen. In one example, both gases are part of the same flow/stream. In another example, each gas is a separate flow/stream provided to the aqueous medium. These gases may be divided for example using separate nozzles that open up into the aqueous medium, frits, membranes within the pipe supplying the gas into the aqueous medium and the like. The oxygen may be free oxygen. According to any aspect of the present invention, ‘a reaction mixture comprising free oxygen’ refers to the reaction mixture comprising elemental oxygen in the form of O₂. The O₂ may be dissolved oxygen in the reaction mixture. In particular, the dissolved oxygen may be in the concentration of 25 ppm (0.000005% vol; 5×10⁻⁶). A skilled person may be capable of using any method known in the art to measure the concentration of dissolved oxygen. In one example, the dissolved oxygen may be measured by Oxygen Dipping Probes (Type PSt6 from PreSens Precision Sensing GmbH, Regensburg, Germany.

Step (b) of the method according to any aspect of the present invention involves contacting the acetate and/or ethanol from step (a) with a third microorganism capable of converting the acetate and/or ethanol to propanol and/or propionic acid. In particular, the third microorganism may be genetically modified to comprise increased expression relative to the wild type cell of enzymes necessary to carry out the methylmalonyl-succinate pathway or the lactate-acrylate pathway.

According to any aspect of the present invention, the first, second and/or third microorganism may be a genetically modified microorganism. The genetically modified cell or microorganism may be genetically different from the wild type cell or microorganism. The genetic difference between the genetically modified microorganism according to any aspect of the present invention and the wild type microorganism may be in the presence of a complete gene, amino acid, nucleotide etc. in the genetically modified microorganism that may be absent in the wild type microorganism. In one example, the genetically modified microorganism according to any aspect of the present invention may comprise enzymes that enable the microorganism to produce propanol and/or propionic acid.

The wild type microorganism relative to the genetically modified microorganism according to any aspect of the present invention may have none or no detectable activity of the enzymes that enable the genetically modified microorganism to produce propanol and/or propionic acid. As used herein, the term ‘genetically modified microorganism’ may be used interchangeably with the term ‘genetically modified cell’. The genetic modification according to any aspect of the present invention may be carried out on the cell of the microorganism.

The phrase “wild type” as used herein in conjunction with a cell or microorganism may denote a cell with a genome make-up that is in a form as seen naturally in the wild. The term may be applicable for both the whole cell and for individual genes. The term “wild type” therefore does not include such cells or such genes where the gene sequences have been altered at least partially by man using recombinant methods.

A skilled person would be able to use any method known in the art to genetically modify a cell or microorganism. According to any aspect of the present invention, the genetically modified cell may be genetically modified so that in a defined time interval, within 2 hours, in particular within 8 hours or 24 hours, it forms at least twice, especially at least 10 times, at least 100 times, at least 1000 times or at least 10000 times more propanol and/or propionic acid than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (propanol and/or propionic acid) in the nutrient medium.

The term “second microorganism” or “third microorganism”, refers to a microorganism that is different from “the first microorganism” according to any aspect of the present invention. The phrase the genetically modified cell has an increased activity, in comparison with its wild type, in enzymes' as used herein refers to the activity of the respective enzyme that is increased by a factor of at least 2, in particular of at least 10, more in particular of at least 100, yet more in particular of at least 1000 and even more in particular of at least 10000.

The phrase “increased activity of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. Similarly, a decreased activity of an enzyme refers to decreased intracellular activity. In one example, the increased expression of an enzyme according to any aspect of the present invention may be 5, 10, 15, 20, 25, 30, 25, 40, 45, 50, 55, 60, 65, 70, 75, 80, 85, 90, 95 or 100% more relative to the expression of the enzyme in the wild type cell. Similarly, the decreased expression of an enzyme according to any aspect of the present invention may be 5, 10, 15, 20, 25, 30, 25, 40, 45, 50, 55, 60, 65, 70, 75, 80, 85, 90, 95 or 100% less relative to the expression of the enzyme in the wild type cell.

The culture medium to be used must be suitable for the requirements of the particular strains. Descriptions of culture media for various microorganisms are given in “Manual of Methods for General Bacteriology”.

All percentages (%) are, unless otherwise specified, mass percent.

With respect to the source of substrates comprising carbon dioxide and/or carbon monoxide, a skilled person would understand that many possible sources for the provision of CO and/or CO₂ as a carbon source exist. It can be seen that in practice, as the carbon source of the present invention any gas or any gas mixture can be used which is able to supply the microorganisms with sufficient amounts of carbon, so that acetate and/or ethanol, may be formed from the source of CO and/or CO₂.

Generally for the cell of the present invention the carbon source comprises at least 50% by weight, at least 70% by weight, particularly at least 90% by weight of CO₂ and/or CO, wherein the percentages by weight—% relate to all carbon sources that are available to the cell according to any aspect of the present invention. The carbon material source may be provided.

Examples of carbon sources in gas forms include exhaust gases such as synthesis gas, flue gas and petroleum refinery gases produced by yeast fermentation or clostridial fermentation. These exhaust gases are formed from the gasification of cellulose-containing materials or coal gasification. In one example, these exhaust gases may not necessarily be produced as by-products of other processes but can specifically be produced for use with the mixed culture of the present invention.

According to any aspect of the present invention, the carbon source may be synthesis gas. Synthesis gas can for example be produced as a by-product of coal gasification. Accordingly, the microorganism according to any aspect of the present invention may be capable of converting a substance which is a waste product into a valuable resource.

In another example, synthesis gas may be a by-product of gasification of widely available, low-cost agricultural raw materials for use with the mixed culture of the present invention to produce substituted and unsubstituted organic compounds.

There are numerous examples of raw materials that can be converted into synthesis gas, as almost all forms of vegetation can be used for this purpose. In particular, raw materials are selected from the group consisting of perennial grasses such as miscanthus, corn residues, processing waste such as sawdust and the like.

In general, synthesis gas may be obtained in a gasification apparatus of dried biomass, mainly through pyrolysis, partial oxidation and steam reforming, wherein the primary products of the synthesis gas are CO, H₂ and CO₂. Syngas may also be a product of electrolysis of CO₂. A skilled person would understand the suitable conditions to carry out electrolysis of CO₂ to produce syngas comprising CO in a desired amount.

Usually, a portion of the synthesis gas obtained from the gasification process is first processed in order to optimize product yields, and to avoid formation of tar. Cracking of the undesired tar and CO in the synthesis gas may be carried out using lime and/or dolomite. These processes are described in detail in for example, Reed, 1981.

Mixtures of sources can be used as a carbon source.

According to any aspect of the present invention, a reducing agent, for example hydrogen may be supplied together with the carbon source. In particular, this hydrogen may be supplied when the C and/or CO₂ is supplied and/or used. In one example, the hydrogen gas is part of the synthesis gas present according to any aspect of the present invention. In another example, where the hydrogen gas in the synthesis gas is insufficient for the method of the present invention, additional hydrogen gas may be supplied.

A skilled person would understand the other conditions necessary to carry out the method according to any aspect of the present invention. In particular, the conditions in the container (e.g. fermenter) may be varied depending on the first, second and third microorganisms used. The varying of the conditions to be suitable for the optimal functioning of the microorganisms is within the knowledge of a skilled person.

In one example, the method according to any aspect of the present invention may be carried out in an aqueous medium with a pH between 5 and 8, 5.5 and 7. The pressure may be between 1 and 10 bar.

The term “contacting”, as used herein, means bringing about direct contact between the cell according to any aspect of the present invention and the medium comprising the carbon source in step (a) and/or the direct contact between the third microorganism and the acetate and/or ethanol from step (a) in step (b). For example, the cell, and the medium comprising the carbon source may be in different compartments in step (a). In particular, the carbon source may be in a gaseous state and added to the medium comprising the cells according to any aspect of the present invention.

In particular, the aqueous medium may comprise the cells and a carbon source comprising CO and/or CO₂ for step (a) to be carried out. More in particular, the carbon source comprising CO and/or CO₂ is provided to the aqueous medium comprising the cells in a continuous gas flow. Even more in particular, the continuous gas flow comprises synthesis gas. These gases may be supplied for example using nozzles that open up into the aqueous medium, frits, membranes within the pipe supplying the gas into the aqueous medium and the like.

The overall efficiency, alcohol productivity and/or overall carbon capture of the method of the present invention may be dependent on the stoichiometry of the CO₂, CO, and H₂ in the continuous gas flow. The continuous gas flows applied may be of composition CO₂ and H₂. In particular, in the continuous gas flow, concentration range of CO₂ may be about 10-50%, in particular 3% by weight and H₂ would be within 44% to 84%, in particular, 64 to 66.04% by weight. In another example, the continuous gas flow can also comprise inert gases like N₂, up to a N₂ concentration of 50% by weight.

The term ‘about’ as used herein refers to a variation within 20 percent. In particular, the term “about” as used herein refers to +/−20%, more in particular, +/−10%, even more in particular, +/−5% of a given measurement or value.

A skilled person would understand that it may be necessary to monitor the composition and flow rates of the streams. Control of the composition of the stream can be achieved by varying the proportions of the constituent streams to achieve a target or desirable composition. The composition and flow rate of the stream can be monitored by any means known in the art. In one example, the system is adapted to continuously monitor the flow rates and compositions of the streams and combine them to produce a single blended substrate stream in a continuous gas flow of optimal composition, and means for passing the optimised substrate stream to the cell according to any aspect of the present invention.

Microorganisms which convert CO₂ and/or CO to acetate and/or ethanol, in particular acetate, as well as appropriate procedures and process conditions for carrying out this metabolic reaction is well known in the art. Such processes are, for example described in WO9800558, WO2000014052 and WO2010115054.

The term “an aqueous solution” or “medium” comprises any solution comprising water, mainly water as solvent that may be used to keep the cell according to any aspect of the present invention, at least temporarily, in a metabolically active and/or viable state and comprises, if such is necessary, any additional substrates. The person skilled in the art is familiar with the preparation of numerous aqueous solutions, usually referred to as media that may be used to keep inventive cells, for example LB medium in the case of E. coli, ATCC1754-Medium may be used in the case of C. Ijungdahlii. It is advantageous to use as an aqueous solution a minimal medium, i.e. a medium of reasonably simple composition that comprises only the minimal set of salts and nutrients indispensable for keeping the cell in a metabolically active and/or viable state, by contrast to complex mediums, to avoid dispensable contamination of the products with unwanted side products. For example, M9 medium may be used as a minimal medium. The cells are incubated with the carbon source sufficiently long enough to produce the desired product, 3HB and variants thereof. For example for at least 1, 2, 4, 5, 10 or 20 hours. The temperature chosen must be such that the cells according to any aspect of the present invention remains catalytically competent and/or metabolically active, for example 10 to 42° C., preferably 30 to 40° C., in particular, 32 to 38° C. in case the cell is a C. Ijungdahlii cell.

In one example, steps (a) and (b) may be carried it out in a single container. In particular, both steps may be carried out under aerobic conditions. This allows for the accumulation of the propanol and/or propionic acid to take place and less media to be used making the reaction more cost effective.

In another example, step (a) and step (b) may be carried out in two different containers. In one example, step (a) may be carried out in fermenter 1 wherein the first and second microorganisms come in contact with the carbon source to produce acetate and/or ethanol. Ethanol and/or acetate may then be brought into contact with a third microorganism in fermenter 2 to produce propanol and/or propionic acid. The propanol and/or propionic acid may then be extracted and the remaining carbon substrate fed back into fermenter 1. A cycle may be created wherein the acetate and/or ethanol produced in fermenter 1 may be regularly fed into fermenter 2, the acetate and/or ethanol in fermenter 2 may be converted to propanol and/or propionic acid and the unreacted carbon source in fermenter 2 fed back into fermenter 1. Oxygen may be added into fermenter 2 to enable the third microorganism to convert acetate to propanol and/or propionic acid. When the remaining carbon source is cycled back from fermenter 2 to fermenter 1, consequently small amounts of oxygen and propanol and/or propionic acid may enter fermenter 1. The presence of these small amounts of oxygen and propanol and/or propionic acid may still allow for the first and second microorganisms to carry out their activity of converting carbon to acetate and/or ethanol.

In another example, the media is being recycled between fermenters 1 and 2. Therefore, the propanol and/or propionic acid produced in fermenter 2 may be fed back into fermenter 1 to accumulate the propanol and/or propionic acid produced according to any aspect of the present invention in the fermenters. In the process of recycling the media, oxygen from fermenter 2 and the propanol and/or propionic acid produced in fermenter 2 are consequently reintroduced into fermenter 1. As can be seen in the examples, the amino acids may not be metabolised by the microorganisms in fermenter 1. Accordingly, the amino acids may accumulate in the media within the two fermenters. Also, the microorganisms in fermenter 1 may be able to continue producing acetate and ethanol in the presence of the oxygen recycled from fermenter 2 into fermenter 1. The accumulated propanol and/or propionic acid may then be extract by means known in the art.

Means of extracting propanol and/or propionic acid according to any aspect of the present invention may include extraction and/or distillation A skilled person may easily identify the most suitable means of extracting propanol and/or propionic acid by simple trial and error. In particular, the methods may include that disclosed in Keshav, A., 2009 and Galaction, A.-I, 2012.

### BRIEF DESCRIPTION OF FIGURES

FIG. 1 is an illustration of the methylmaionyl-succinate pathway used by anaerobic microorganisms for C3 (propionate) production (Source: WO2014/140336).

FIG. 2 is an illustration of the lactate-acrylate pathway used by anaerobic microorganisms for C3 (propionate/propanol) production (Source: WO2014/140336).

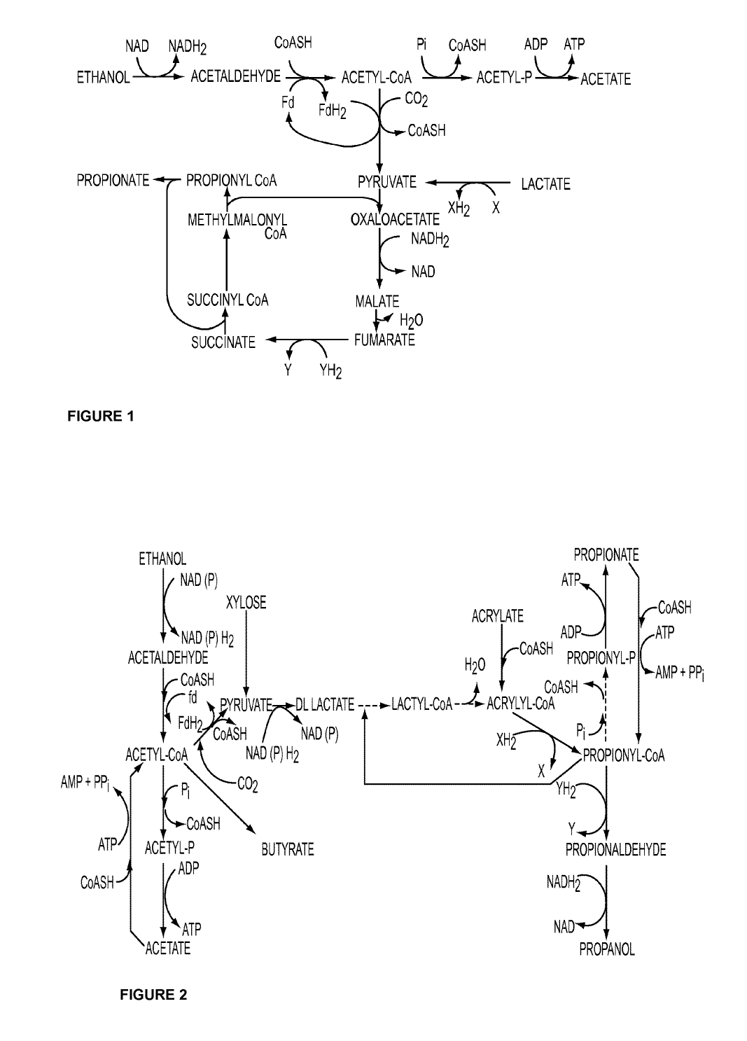

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example I

Production of Propionic Acid and Propanol on Synthesis Gas with Oxygen with Clostridium autoethanogenum and Clostridium neopropionicum

For the biotransformation of hydrogen and carbon dioxide to propionic acid and propanol the homoacetogenic bacterium Clostridium autoethanogenum was cultivated on synthesis gas in combination with Clostridium neopropionicum in a co-cultivation phase. All cultivation steps were carried out under anaerobic or microaerophile conditions in pressure-resistant glass bottles that can be closed airtight with a butyl rubber stopper.

For the cultivation of C. autoethanogenum 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl_{2×2} H₂O; 20 mg/L nitrilotriacetic acid; 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO_{4×2} H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 64 h till OD₆₀₀ₙₘ of 0.36. The gas was discharged into the medium through a sparger with a pore size of 10 μm, which was mounted in the center of the reactors. Then the cell suspension was centrifuged, washed with fresh ATCC1754-medium and centrifuged again. For the main culture of C. autoethanogenum 500 ml ATCC1754-medium with additional 400 mg/L L-cysteine-hydrochlorid and 400 mg/L Na₂S×9H₂O were inoculated with washed cells from the first preculture to an OD₆₀₀ₙₘ of 0.1. The chemolithoautotrophic cultivation was carried out in a 1 L pressure-resistant glass bottle at 37° C., 100 rpm and a ventilation rate of 1 L/h with a premixed gas with 66.85% H₂, 33% CO₂, 0.15% O₂ in an open water bath shaker for 51 h till OD₆₀₀ₙₘ of 0.19 and pH 5.7. The gas was discharged into the medium through a sparger with a pore size of 10 μm, which was mounted in the center of the reactors.

For the first preculture of C. neopropionicum 2×100 ml DSMZ318 medium (pH 7.4; 0.61 g/l NaCl, 0.047 g/l MgCl₂, 0.30 g/l KH₂PO₄, 1.00 g/l NH₄Cl, 0.081 g/l CaCl_{2×2} H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl_{3×6} H₂O, 1 mg/L MnCl_{2×4} H₂O, 0.24 mg/L CoCl_{2×6} H₂O, 1 mg/L ZnCl₂, 0.25 mg/L CuCl_{2×2} H₂O, 0.1 mg/L H₃BO₃, 0.24 mg/L Na₂MoO_{4×2} H₂O, 1.2 mg/L NiCl_{2×6} H₂O, 0.26 mg/L Na₂SeO_{3×5} H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B₁₂, 0.05 mg/L p-aminobenzoate, 0.05 mg/L lipoic acid, 0.25 g/L cysteine-HCl×H₂O) in a 250 ml bottle were inoculated with 5 ml of a frozen cryoculture and flushed with a premixed gas with 67% H₂, 33% CO₂ to an overpressure of 0.8 bar. The culture was incubated at 30° C. and 100 rpm in an open water bath shaker for 19 h till an OD₆₀₀ₙₘ>0.14.

For a second preculture of Clostridium neopropionicum 5×200 ml of fresh DSMZ318 medium in a 500 ml bottle were inoculated with centrifuged cells from the first preculture to an OD₆₀₀ₙₘ of 0.02 and flushed with a premixed gas with 67% H₂, 33% CO₂ to an overpressure of 0.8 bar. This growing culture was incubated at 30° C. and 100 rpm in an open water bath shaker for 24 h till an OD₆₀₀ₙₘ>0.26. Then the cell suspension was centrifuged, washed with fresh ATCC1754-medium and centrifuged again.

For the co-cultivation culture, as many washed cells from the second preculture of C. neopropionicum as necessary for an OD₆₀₀ₙₘ of 0.2 were added to the continuously aerated main culture of C. autoethanogenum after 51 h of cultivation. The cultivation was carried out in a 1 L pressure-resistant glass bottle at 37° C., 100 rpm and a ventilation rate of 1 L/h with a premixed gas with 66.85% H₂, 33% CO₂, 0.15% O₂ in an open water bath shaker for another 41 h. At the beginning the pH was set to 6.7 with 140 g/l KOH and then the co-cultivation was carried out without pH control.

During cultivation several 5 mL samples were taken to determinate OD₆₀₀ₙₘ, pH und product formation. The determination of the product concentrations was performed by LCMS and semiquantitative ¹H-NMR spectroscopy. As an internal quantification standard sodium trimethylsilylpropionate (T(M)SP) was used.

During the co-cultivation the concentration of acetate increased from 0.79 g/L to 1.83 g/L, for propionate from 0.00 g/L to 0.23 g/L, for propanol from 0 to 19 mg/L, for butyrate from 0 to 14 mg/L, and for formate from 32 mg/L to 335 mg/L. The concentration of ethanol decreased from 47 mg/L to 25 mg/L during this time.

### Example 2

### Culture of Clostridium Ijungdahlii in Log Phase in the Presence of Synthesis Gas Comprising CO₂ and 0.15% Oxygen

C. jungdahlii was fed H₂ and CO₂ out of the feed-through gas phase and formed acetate and ethanol. For the cultivation, pressure-resistant glass bottle that can be sealed airtight with a butyl rubber stopper were used. All cultivation steps, where C. Ijungdahlii cells were involved were carried out under anaerobic conditions.

For cell culture of C. Ijungdahlii 5 mL Cryoculture was cultured anaerobically in 500 ml of medium (ATCC1754 medium: pH 6.0; 20 g/L MES; 1 g/L yeast extract, 0.8 g/L NaCl, 1 g/L NH₄Cl, 0.1 g/L KCl, 0.1 g/L KH₂PO₄, 0.2 g/L MgSO_{4×7} H₂O; 0.02 g/L CaCl_{2×2}H₂O; 20 mg/L nitrilotriacetic acid 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO_{4×2} H₂O; 20 μg/L d-Biotin, 20 μg/L folic acid, 100 g/L pyridoxine-HCl; 50 μg/L thiamine-HCl×H₂O; 50 μg/L riboflavin; 50 μg/L nicotinic acid, 50 μg/L Ca-pantothenate; 1 μg/L vitamin B₁₂; 50 μg/L p-aminobenzoate; 50 μg/L lipoic acid, approximately 67.5 mg/L NaOH) with about 400 mg/L L-cysteine hydrochloride and 400 mg/L Na₂S×9H₂O. Cultivation was carried chemolithoautotrophically in a flameproof 1 L glass bottle with a premixed gas mixture composed of 67% H₂, 33% CO₂ in an open water bath shaker at 37° C., 100 rpm and a fumigation of 3 L/h for 72 h. The gas entry into the medium was carried out by a filter with a pore size of 10 microns, and was mounted in the middle of the reactor, at a gassing tube. The cells were centrifuged, washed with 10 ml ATCC medium and centrifuged again.

For the main culture many washed cells from the growth culture of C. Ijungdahlii were transferred into 500 mL of ATCC medium with about 400 mg/L L-cysteine hydrochloride and grown to an OD₆₀₀ of 0.1. Cultivation was carried out in a pressure-resistant 1 L glass bottle with a premixed gas mixture composed of 66.85% H₂, 33% CO₂, 0.15% O₂ in an open water bath shaker at 37° C., 150 rpm and with aeration of 1 L/h for 45 h. The gas entry into the medium was carried out by a filter with a pore size of 10 microns, which was placed in the middle of the reactors. When sampling each 5 ml sample was removed for determination of OD₆₀₀ nm, pH and the product range. The determination of the product concentration was performed by semi-quantitative 1H-NMR spectroscopy. As an internal quantification standard sodium trimethylsilylpropionate served (T (M) SP).

There was significant cell growth shown during the cultivation period, evidenced by an increase in OD₆₀₀ nm of 0.10 to 0.54, corresponding to a growth rate p=0.037 h⁻¹. The concentration of acetate increased at the same time from 9.6 mg/L to 3,304 mg/L and the concentration of ethanol from 2.2 mg/L to 399 mg/L.

### Example 3

### Culture of Clostridium Jungdahlii in Log Phase in the Presence of Synthesis Gas Comprising CO and 0.1% Oxygen

C. Ijungdahlii was autotrophically cultivated in complex medium with synthesis gas, consisting of CO, H₂ and CO₂ in the presence of oxygen in order to produce acetate and ethanol.

A complex medium was used consisting of 1 g/L NH₄Cl, 0.1 g/L KCl, 0.2 g/L MgSO_{4×7} H₂O, 0.8 g/L NaCl, 0.1 g/L KH₂PO₄, 20 mg/L CaCl_{2×2} H₂O, 20 g/L MES, 1 g/L yeast extract, 0.4 g/L L-cysteine-HCl, 0.4 g/L Na₂S×9H₂O, 20 mg/L nitrilotriacetic acid, 10 mg/L MnSO₄×H₂O, 8 mg/L (NH₄)₂Fe(SO₄)_{2×6} H₂O, 2 mg/L CoCl_{2×6} H₂O, 2 mg/L ZnSO_{4×7} H₂O, 0.2 mg/L CuCl_{2×2} H₂O, 0.2 mg/L Na₂MoO_{4×2} H₂O, 0.2 mg/L NiCl_{2×6} H₂O, 0.2 mg/L Na₂SeO₄, 0.2 mg/L Na₂WO_{4×2} H₂O, 20 μg/L biotin, 20 μg/L folic acid, 100 μg/L pyridoxine-HCl, 50 μg/L thiamine-HCl×H₂O, 50 μg/L riboflavin, 50 μg/L nicotinic acid, 50 μg/L Ca-pantothenoic acid, 1 μg/L vitamine B12, 50 μg/L p-aminobenzoic acid, 50 μg/L lipoic acid.

The autotrophic cultivation was performed in 500 mL medium in a 1 L serum bottle that was continuously gassed with synthesis gas consisting of 67.7% CO, 3.5% H₂ and 15.6% CO₂ at a rate of 3.6 L/h. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 120 min⁻¹.

pH was not controlled.

At the beginning of the experiment, C. Ijungdahlii was inoculated with an OD₆₀₀ of 0.1 with autotrophically grown cells on H₂/CO₂. Therefore, C. Ijungdahlii was grown in complex medium under continuous gassing with synthesis gas consisting of 67% H₂ and 33% CO₂ at a rate of 3 L/h in 1 L serum bottles with 500 mL complex medium. Above described medium was also used for this cultivation. The gas was introduced into the liquid phase by a microbubble disperser with a pore diameter of 10 μm. The serum bottle was continuously shaken in an open water bath Innova 3100 from New Brunswick Scientific at 37° C. and a shaking rate of 150 min⁻¹. The cells were harvested in the logarithmic phase with an OD₆₀₀ of 0.49 and a pH of 5.03 by anaerobic centrifugation (4500 min⁻¹, 4300 g, 20° C., 10 min). The supernatant was discarded and the pellet was resuspended in 10 mL of above described medium. This cell suspension was then used to inoculate the cultivation experiment. Gas phase concentration of carbon monoxide was measured sampling of the gas phase and offline analysis by an gas chromatograph GC 6890N of Agilent Technologies Inc. with an thermal conductivity detector. Gas phase concentration of oxygen was measured by a trace oxygen dipping probe from PreSens Precision Sensing GmbH. Oxygen concentration was measured by fluorescence quenching, whereas the degree of quenching correlates to the partial pressure of oxygen in the gas phase. Oxygen measurement indicated a concentration of 0.1% vol of O₂ in the used synthesis gas.

During the experiment samples of 5 mL were taken for the determination of OD₆₀₀, pH and product concentrations. The latter were determined by quantitative ¹H-NMR-spectroscopy.

After inoculation of C. Ijungdahlii, cells began to grow with a growth rate μ of 0.062 h⁻¹ and continuously produced acetate up to a concentration of 6.2 g/L after 94.5 hours. Concomitant to the production of acetate, ethanol was produced in a lower rate compared to the production of acetate up to a concentration of 1 g/L after 94.5 hours.

TABLE 1
results of example 4

NMR-analytics

| Process | | | Acetate, | Ethanol, |
|---|---|---|---|---|
| time, h | pH | OD600 | mg/L | mg/L |
| 0.0 | 6.15 | 0.10 | 18 | n.d. |
| 18.0 | 5.97 | 0.69 | 973 | 97 |
| 42.5 | 5.20 | 1.50 | | |
| 66.0 | 4.67 | 1.95 | 5368 | 966 |
| 94.5 | 4.54 | 1.77 | 6187 | 1070 |

(n.d. = not detected)

### Example 4

Growth and Acetate Production by Clostridium Ijungdahlii on Synthesis Gas with 2% Oxygen

For the biotransformation of hydrogen and carbon dioxide to acetic acid the homoacetogenic bacterium Clostridium Ijungdahlii was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO_{4×7} H₂O; 0.02 g/L CaCl_{2×2} H₂O; 20 mg/L nitrilotriacetic acid; 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO_{4×2} H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock of C. Ijungdahlii. The chemolithoautotrophic cultivation was carried out in a 1 L pressure-resistant glass bottle at 37° C., 100 rpm and a ventilation rate of 3 L/h with a premixed gas with 67% H₂, 33% CO₂ in an open water bath shaker for 72 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was washed with 10 ml medium and centrifuged again. For the main culture, as many washed cells from the preculture as necessary for an OD₆₀₀ₙₘ of 0.1 were transferred in 200 mL medium with additional 400 mg/L L-cysteine-hydrochlorid. The chemolithoautotrophic cultivation was carried out in a 250 mL pressure-resistant glass bottles at 37° C., 150 rpm and a ventilation rate of 1 L/h with a premixed gas with 65% H₂, 33% CO₂, 2% O₂ in an open water bath shaker for 47 h. The gas was discharged into the medium through a sparger with a pore size of 10 μm, which was mounted in the center of the reactors. Culturing was carried out with no pH control. During cultivation several 5 mL samples were taken to determinate OD₆₀₀ₙₘ, pH und product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed by an increase of the OD₆₀₀ₙₘ from 0.11 to 0.32, which correlates with a growth rate of p=0.022 h⁻¹. The concentration of acetate increased from 8 mg/L to 91 mg/L, an increase of the ethanol concentration was not observed. Over the cultivation period the dissolved oxygen concentration varied between 0.06 and 0.15 mg/L.

In a similar technical setting with the same parameters (medium composition, volume, bottle, gas, ventilation rate, temperature, shaking frequency), but without cells in the medium, a dissolved oxygen concentration of 0.50 mg/L was measured.

### Example 5

Growth and Production of Acetate and Other Compounds by Clostridium carboxidivorans on Synthesis Gas with 0.05% Oxygen

For the biotransformation of hydrogen and carbon dioxide to acetic acid and other compounds the homoacetogenic bacterium Clostridium carboxidivorans was cultivated on synthesis gas with oxygen. All cultivation steps were carried out under anaerobic conditions in pressure-resistant glass bottles that can be closed airtight with a butyl rubber stopper.

For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO_{4×7} H₂O; 0.02 g/L CaCl_{2×2} H₂O; 20 mg/L nitrilotriacetic acid; 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO_{4×2} H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochloride and 400 mg/L Na₂S×9H₂O were inoculated with 5 mL of a frozen cryo stock of C. carboxidivorans. The chemolithoautotrophic cultivation was carried out in a 1 L pressure-resistant glass bottle at 37° C., 100 rpm and a ventilation rate of 3 L/h with a premixed gas with 60% H₂, 20% CO₂, and 20% CO in an open water bath shaker for 71 h. The gas was discharged into the medium through a sparger with a pore size of 10 μm, which was mounted in the center of the reactors. Culturing was carried out with no pH control.

After the precultivation, the cell suspension was centrifuged (10 min, 4200 rpm) and the pellet was resuspended in fresh medium. For the main culture, as many cells from the preculture as necessary for an OD₆₀₀ₙₘ of 0.2 were transferred in 200 mL complex medium (ATCC1754) and parallel in 200 ml mineral medium (DM4-medium: pH=6.00, 0.5 g/L MgCl_{2×6} H₂O, 0.2 g/L CaCl₂×2 H₂O, 15 mg/L FeCl_{2×4} H₂O, 2 g/L (NH₄)H₂PO₄, 0.2 g/L NaCl, 0.15 g/L KCl, 3 mg/L H₃BO₃, 2 mg/L CoCl_{2×6} H₂O, 1 mg/L ZnSO_{4×7} H₂O, 300 μg/L Na₂MoO_{4×2} H₂O, 300 μg/L MnSO₄×H₂O, 200 μg/L NiCl_{2×6} H₂O, 100 μg/L CuCl_{2×2} H₂O, 100 μg/L Na₂SeO₃, 106 μg/L d-biotin, 5 μg/L folic acid, 2.5 μg/L pyridoxine-HCl, 266 μg/L thiamine-HCl, 12.5 μg/L riboflavin, 12.5 μg/L nicotinic acid, 413 μg/L Ca-pantothenate, 12.5 μg/L vitamin B₁₂, 12.5 μg/L p-aminobenzoate, 15.0 μg/L lipoic acid, approx. 1.3 g/L KOH), with additional 400 mg/L L-cysteine-hydrochloride each. The chemolithoautotrophic cultivation was carried out in a 1 L pressure-resistant glass bottle at 37° C., 150 rpm and a ventilation rate of 1 L/h with a premixed gas with 66.95% H₂, 33% CO₂, and 0.05% O₂ in an open water bath shaker for 40 h. The gas was discharged into the head space through a sparger with a pore size of 10 μm, which was mounted in the center of the reactors. Culturing was carried out with no pH control. During cultivation several 5 mL samples were taken to determinate OD₆₀₀ₙₘ, pH und product formation. The determination of the product concentrations was performed by semiquantitative 1H-NMR spectroscopy. As an internal quantification standard sodium trimethylsilylpropionate (T(M)SP) was used. Also the dissolved oxygen in the cultivation medium was measured online by oxygen dipping probes (PSt6 with Oxy4Trace, Presens, Germany).

During the cultivation period cell growth was observed in complex medium by an increase of the OD₆₀₀ₙₘ from 0.20 to 0.36, which correlates with a growth rate of p=0.015 h⁻¹. In mineral medium, the OD₆₀₀ₙₘ decreased from 0.20 to 0.19. In complex medium the concentration of acetate increased from 29 mg/L to 280 mg/L, for ethanol from 3 mg/L to 82 mg/L, for butyrate from 0 mg/L to 29 mg/L and for butanol from 0 mg/L to 10 mg/L. In mineral medium the concentration of acetate increased from 25 mg/L to 110 mg/L, for ethanol from 3 mg/L to 5 mg/L and for butyrate from 0 mg/L to 2 mg/L. Over the whole cultivation period the dissolved oxygen concentration in both cultures was 0.00 mg/L. In a similar technical setting with the same parameters (medium composition, volume, bottle, gas, ventilation rate, temperature, shaking frequency), but without cells in the medium, a dissolved oxygen concentration of 0.01 mg/L was measured in both media.

### Example 6

Growth and Production of Acetate by Acetobacterium Woodii on Synthesis Gas with Oxygen

For the biotransformation of hydrogen and carbon dioxide to acetic acid the homoacetogenic bacterium Acetobacterium woodii is cultivated on synthesis gas with oxygen. All cultivation steps are carried out under anaerobic conditions in pressure-resistant glass bottles that can be closed airtight with a butyl rubber stopper. For the preculture 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO_{4×7} H₂O; 0.02 g/L CaCl_{2×2} H₂O; 20 mg/L nitrilotriacetic acid; 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO₄×2 H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochloride and 400 mg/L Na₂S×9H₂O are inoculated with 5 mL of a frozen cryo stock of A. woodii. The chemolithoautotrophic cultivation is carried out in a 1 L pressure-resistant glass bottle at 37° C., 100 rpm and a ventilation rate of 3 L/h with a premixed gas with 67% H₂, 33% CO₂ in an open water bath shaker for 72 h. The gas is discharged into the medium through a sparger with a pore size of 10 μm, which is mounted in the center of the reactors. Culturing is carried out with no pH control.

After the precultivation, the cell suspension is centrifuged (10 min, 4200 rpm) and the pellet is resuspended in fresh medium. For the main culture, as many cells from the preculture as necessary for an OD₆₀₀ₙₘ of 0.1 are transferred in 500 mL medium with additional 400 mg/L L-cysteine-hydrochlorid. The chemolithoautotrophic cultivation is carried out in a 1 L pressure-resistant glass bottle at 37° C., 150 rpm and a ventilation rate of 1 L/h with a premixed gas with 66.95% H₂, 33% CO₂, 0.05% O₂ in an open water bath shaker for 41 h. The gas is discharged into the medium through a sparger with a pore size of 10 μm, which is mounted in the center of the reactors. Culturing is carried out with no pH control. During cultivation several 5 mL samples are taken to determinate OD₆₀₀ₙₘ, pH und product formation. The determination of the product concentrations is performed by semiquantitative 1H-NMR spectroscopy. As an internal quantification standard sodium trimethylsilylpropionate (T(M)SP) is used. Also the dissolved oxygen in the cultivation medium is measured online by oxygen dipping probes (PSt6 with Oxy4Trace, Presens, Germany).

During the cultivation period cell growth is observed by an increase of the OD₆₀₀ₙₘ. Also the concentration of acetate increases.

In a similar technical setting with the same parameters (medium composition, volume, bottle, gas, ventilation rate, temperature, shaking frequency), but without cells in the medium, a dissolved oxygen concentration of 0.01 mg/L is measured.

### Example 7

Production of Propionic Acid and Propanol with Clostridium neopropionicum on Synthesis Gas

For the biotransformation of ethanol and carbon dioxide to propionic acid and propanol the bacterium Clostridium neopropionicum was cultivated with ethanol and a gas atmosphere with carbon dioxide. All cultivation steps were carried out under anaerobic conditions in pressure-resistant glass bottles that can be closed airtight with a butyl rubber stopper.

For the first preculture of C. neopropionicum 2×100 ml DSMZ318 medium (pH 7.4; 0.61 g/l NaCl, 0.047 g/l MgCl₂, 0.30 g/l KH₂PO₄, 1.00 g/l NH₄Cl, 0.081 g/l CaCl_{2×2} H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl_{3×6} H₂O, 1 mg/L MnCl_{2×4} H₂O, 0.24 mg/L CoCl_{2×6} H₂O, 1 mg/L ZnCl₂, 0.25 mg/L CuCl_{2×2} H₂O, 0.1 mg/L H₃BO₃, 0.24 mg/L Na₂MoO_{4×2} H₂O, 1.2 mg/L NiCl_{2×6} H₂O, 0.26 mg/L Na₂SeO_{3×5} H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B12, 0.05 mg/L p-aminobenzoate, 0.05 mg/L lipoic acid, 0.25 g/L cysteine-HCl×H₂O) in 250 ml pressure resistant bottles were inoculated with 5 ml of a frozen cryoculture and flushed with a premixed gas with 67% H₂, 33% CO₂ to an overpressure of 0.8 bar. The cultures were incubated at 30° C. and 100 rpm in an open water bath shaker for 24 h to an OD₆₀₀ₙₘ 0.14-0.16.

For a second preculture of C. neopropionicum 3×200 ml of fresh DSMZ318 medium in 500 ml pressure resistant bottles were inoculated with centrifuged cells from the first preculture to an OD₆₀₀ₙₘ of 0.03 and flushed with a premixed gas with 67% H₂, 33% CO₂ to an overpressure of 0.8 bar. These growing culture were incubated at 30° C. and 100 rpm in an open water bath shaker for 22 h to an OD₆₀₀ₙₘ 0.24.

For the main culture, as many centrifuged cells from the second preculture of C. neopropionicum as necessary for an OD₆₀₀ₙₘ of 0.2 were added to 200 ml of fresh LM33 mineral medium (pH=6.8, 10 g/L ethanol, 1 g/L NaOH, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO_{4×7} H₂O, 5 mg/L MnSO₄×H₂O, 1 mg/L FeSO_{4×7} H₂O, 8 mg/L Fe(SO₄)₂(NH₄)_{2×6} H₂O, 2 mg/L CoCl_{2×6} H₂O, 2 mg/L ZnSO_{4×7} H₂O, 200 μg/L CuCl_{2×2} H₂O, 200 μg/L KAI(SO₄)_{2×12} H₂O, 3 mg/L H₃BO₃, 300 μg/L Na₂MoO_{4×2} H₂O, 200 μg/L Na₂SeO₃, 200 μg/L NiCl_{2×6} H₂O, 200 μg/L Na₂WO₄×6 H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B₁₂; 500 μg/L p-aminobenzoate; 500 μg/L lipoic acid, 10 mg/L FeCl₃, aerated for 30 min with a premixed gas with 67% H₂ and 33% CO₂). The cultivation was carried out in a 500 mL pressure-resistant glass bottle at 30° C., 100 rpm and an overpressure of 0.8 bar of a premixed gas with 67% H₂, 33% CO₂ in an open water bath shaker for 114 h. The pH was held at 6.8 by automatic addition of NaOH solution (100 g/L).

During cultivation several 5 mL samples were taken to determinate OD₆₀₀ₙₘ, pH und product formation. The determination of the product concentrations was performed by semi-quantitative ¹H-NMR spectroscopy. As an internal quantification standard sodium trimethylsilylpropionate (T(M)SP) was used.

During the cultivation the concentration of propionate increased from 0.03 g/L to 2.65 g/L, for propanol from 0.007 to 0.45 g/L, for butyrate from 0.003 to 0.38 g/L and for lactate from 0 g/L to 0.17 g/L. The concentration of ethanol decreased from 10.4 g/L to 5.7 g/L during this time.

### Example 8

Production of Propionic Acid and Propanol with Clostridium neopropionicum on Synthesis Gas with 0.05% Oxygen

For the biotransformation of ethanol and carbon dioxide to propionic acid and propanol the bacterium Clostridium neopropionicum was cultivated with ethanol and a gas atmosphere with carbon dioxide. All cultivation steps were carried out under anaerobic conditions in pressure-resistant glass bottles that can be closed airtight with a butyl rubber stopper.

For the first preculture of C. neopropionicum 2×100 ml DSMZ318 medium (pH 7.4; 0.61 g/l NaCl, 0.047 g/l MgCl₂, 0.30 g/l KH₂PO₄, 1.00 g/l NH₄Cl, 0.081 g/l CaCl_{2×2} H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl_{3×6} H₂O, 1 mg/L MnCl_{2×4} H₂O, 0.24 mg/L CoCl_{2×6} H₂O, 1 mg/L ZnCl₂, 0.25 mg/L CuCl_{2×2} H₂O, 0.1 mg/L H₃BO₃, 0.24 mg/L Na₂MoO_{4×2} H₂O, 1.2 mg/L NiCl_{2×6} H₂O, 0.26 mg/L Na₂SeO_{3×5} H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B12, 0.05 mg/L p-aminobenzoate, 0.05 mg/L lipoic acid, 0.25 g/L cysteine-HCl×H₂O) in 250 ml pressure resistant bottles were inoculated with 5 ml of a frozen cryoculture and flushed with a premixed gas with 67% H₂, 33% CO₂ to an overpressure of 0.8 bar. The cultures were incubated at 30° C. and 100 rpm in an open water bath shaker for 24 h to an OD₆₀₀ₙₘ 0.14-0.16.

For a second preculture of C. neopropionicum 3×200 ml of fresh DSMZ318 medium in 500 ml pressure resistant bottles were inoculated with centrifuged cells from the first preculture to an OD₆₀₀ₙₘ of 0.03 and flushed with a premixed gas with 67% H₂, 33% CO₂ to an overpressure of 0.8 bar. These growing culture were incubated at 30° C. and 100 rpm in an open water bath shaker for 22 h to an OD₆₀₀ₙₘ 0.24.

For the main culture, as many centrifuged cells from the second preculture of C. neopropionicum as necessary for an OD₆₀₀ₙₘ of 0.2 were added to 200 ml of fresh LM33 mineral medium (pH=6.8, 10 g/L ethanol, 1 g/L NaOH, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO_{4×7} H₂O, 5 mg/L MnSO₄×H₂O, 1 mg/L FeSO_{4×7} H₂O, 8 mg/L Fe(SO₄)₂(NH₄)_{2×6} H₂O, 2 mg/L CoCl_{2×6} H₂O, 2 mg/L ZnSO_{4×7} H₂O, 200 μg/L CuCl_{2×2} H₂O, 200 μg/L KAI(SO₄)_{2×12} H₂O, 3 mg/L H₃BO₃, 300 μg/L Na₂MoO_{4×2} H₂O, 200 μg/L Na₂SeO₃, 200 μg/L NiCl_{2×6} H₂O, 200 μg/L Na₂WO₄×6 H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B₁₂; 500 μg/L p-aminobenzoate; 500 μg/L lipoic acid, 10 mg/L FeCl₃, aerated for 30 min with a premixed gas with 67% H₂, 33% CO₂, 0.05% O₂). The cultivation was carried out in a 500 mL pressure-resistant glass bottle at 30° C., 100 rpm and an overpressure of 0.8 bar of a premixed gas with 67% H₂, 33% CO₂, 0.05% O₂ in an open water bath shaker for 114 h. The pH was held at 6.8 by automatic addition of NaOH solution (100 g/L).

During cultivation several 5 mL samples were taken to determinate OD₆₀₀ₙₘ pH und product formation. The determination of the product concentrations was performed by semi-quantitative ¹H-NMR spectroscopy. As an internal quantification standard sodium trimethylsilylpropionate (T(M)SP) was used.

During the cultivation the concentration of propionate increased from 0.03 g/L to 3 g/L, for propanol from 0.009 to 0.4 g/L, for butyrate from 0.003 to 0.44 g/L and for lactate from 0 g/L to 0.19 g/L. The concentration of ethanol decreased from 10.8 g/L to 5.2 g/L during this time.

### Example 9

Production of Propionic Acid and Propanol with Clostridium neopropionicum on Synthesis Gas with Spent Medium from a Gas Fermentation with C. autoethanogenum

For the biotransformation of hydrogen and carbon dioxide to propanol the homoacetogenic bacterium Clostridium autoethanogenum was cultivated on synthesis gas with a subsequent cultivation step with Clostridium neopropionicum. All cultivation steps were carried out under anaerobic conditions in pressure-resistant glass bottles that can be closed airtight with a butyl rubber stopper.

For the cultivation of C. autoethanogenum 500 ml medium (ATCC1754-medium: pH=6.0; 20 g/L MES; 1 g/L yeast extract, 0.8 g/L NaCl; 1 g/L NH₄Cl; 0.1 g/L KCl; 0.1 g/L KH₂PO₄; 0.2 g/L MgSO₄×7 H₂O; 0.02 g/L CaCl_{2×2} H₂O; 20 mg/L nitrilotriacetic acid; 10 mg/L MnSO₄×H₂O; 8 mg/L (NH₄)₂Fe(SO₄)_{2×6} H₂O; 2 mg/L CoCl_{2×6} H₂O; 2 mg/L ZnSO_{4×7} H₂O; 0.2 mg/L CuCl_{2×2} H₂O; 0.2 mg/L Na₂MoO_{4×2} H₂O; 0.2 mg/L NiCl_{2×6} H₂O; 0.2 mg/L Na₂SeO₄; 0.2 mg/L Na₂WO_{4×2} H₂O; 20 μg/L d-biotin; 20 μg/L folic acid; 100 μg/L pyridoxine-HCl; 50 μg/L thiamine-HCl×H₂O; 50 μg/L riboflavin; 50 μg/L nicotinic acid; 50 μg/L Ca-pantothenate; 1 μg/L vitamin B₁₂; 50 μg/L p-aminobenzoate; 50 μg/L lipoic acid; approx. 67.5 mg/L NaOH) with additional 400 mg/L L-cysteine-hydrochlorid and 400 mg/L Na₂S×9H₂O were inoculated with 5 mL of a frozen cryo stock. The chemolithoautotrophic cultivation was carried out in 1 L pressure-resistant glass bottle at 37° C., 100 rpm and a ventilation rate of 1 L/h with a premixed gas with 67% H₂ and 33% CO₂ in an open water bath shaker for 70 h till OD₆₀₀ₙₘ of 0.56. The gas was discharged into the medium through a sparger with a pore size of 10 μm, which was mounted in the center of the reactors. Then the cell suspension was centrifuged.

For the main culture of C. autoethanogenum 500 ml LM33 mineral medium (pH=5.9, 1.3 g/L KOH, 0.5 g/L MgCl₂, 0.21 g/L NaCl, 0.135 g/L CaCl₂×2H₂O, 2.65 g/L NaH₂PO₄×2H₂O, 0.5 g/L KCl, 2.5 g/L NH₄Cl, 15 mg/L nitrilotriacetic acid, 30 mg/L MgSO_{4×7} H₂O, 5 mg/L MnSO₄×H₂O, 1 mg/L FeSO_{4×7} H₂O, 8 mg/L Fe(SO₄)₂(NH₄)_{2×6} H₂O, 2 mg/L CoCl_{2×6} H₂O, 2 mg/L ZnSO_{4×7} H₂O, 200 μg/L CuCl_{2×2} H₂O, 200 μg/L KAI(SO₄)_{2×12} H₂O, 3 mg/L H₃BO₃, 300 μg/L Na₂MoO_{4×2} H₂O, 200 μg/L Na₂SeO₃, 200 μg/L NiCl_{2×6} H₂O, 200 μg/L Na₂WO_{4×6} H₂O, 200 μg/L d-biotin, 200 μg/L folic acid, 100 μg/L pyridoxine-HCl, 500 μg/L thiamine-HCl; 500 μg/L riboflavin; 500 μg/L nicotinic acid; 500 μg/L Ca-pantothenate; 500 μg/L vitamin B₁₂; 500 μg/L p-aminobenzoate; 500 μg/L lipoic acid, 10 mg/L FeCl₃, aerated for 30 min with a premixed gas with 67% H₂ and 33% CO₂ with additional 500 mg/L L-cysteine-hydrochlorid and 0.5 mg/L resazurin) were inoculated with cells from the first preculture to an OD₆₀₀ₙₘ of 0.09. The cultivation was carried out in a 1 L pressure-resistant glass bottle at 37° C., 150 rpm in a water bath shaker, with manual pH adjustment to pH 5.8-5.9 at 19, 42, 67 and 93 h of incubation using 100 g/L NaOH. Premixed gas with 67% H₂, 33% CO₂ was discharged at 1 L/h into the medium through a sparger with a pore size of 10 μm, which was mounted in the center of the reactor. During 187 h 3.3 g/L ethanol and 7.9 g/L acetate were produced. After cultivation, the medium was sterile-filtered and used for the main culture of Clostridium neopropionicum.

For the biotransformation of ethanol and carbon dioxide to propionic acid and propanol the bacterium Clostridium neopropionicum was cultivated with ethanol and a gas atmosphere with carbon dioxide. All cultivation steps were carried out under anaerobic conditions in pressure-resistant glass bottles that can be closed airtight with a butyl rubber stopper.

For the first preculture of C. neopropionicum 2×100 ml DSMZ318 medium (pH 7.4; 0.61 g/l NaCl, 0.047 g/l MgCl₂, 0.30 g/l KH₂PO₄, 1.00 g/l NH₄Cl, 0.081 g/l CaCl_{2×2} H₂O, 0.5 g/l yeast extract, 0.5 g/l BBL Trypticase Peptone, 4 g/L KHCO₃, 1.026 g/L ethanol, 0.5 mg/l resazurin, 128 mg/L nitrilotriacetic acid, 135 mg/L FeCl_{3×6} H₂O, 1 mg/L MnCl_{2×4} H₂O, 0.24 mg/L CoCl_{2×6} H₂O, 1 mg/L ZnCl₂, 0.25 mg/L CuCl_{2×2} H₂O, 0.1 mg/L H₃BO₃, 0.24 mg/L Na₂MoO_{4×2} H₂O, 1.2 mg/L NiCl_{2×6} H₂O, 0.26 mg/L Na₂SeO_{3×5} H₂O, 0.02 mg/L biotin, 0.02 mg/L folic acid, 0.1 mg/L pyridoxin-HCl, 0.05 mg/L thiamine-HCl×H₂O, 0.05 mg/L riboflavin, 0.05 mg/L nicotinic acid, 0.05 mg/L D-Ca-pantothenate, 1 μg/L vitamin B12, 0.05 mg/L p-aminobenzoate, 0.05 mg/L lipoic acid, 0.25 g/L cysteine-HCl×H₂O) in 250 ml pressure resistant bottles were inoculated with 5 ml of a frozen cryoculture and flushed with a premixed gas with 67% H₂, 33% CO₂ to an overpressure of 0.8 bar. The cultures were incubated at 30° C. and 100 rpm in an open water bath shaker for 24 h to an OD₆₀₀ₙₘ 0.14-0.16.

For a second preculture of C. neopropionicum 3×200 ml of fresh DSMZ318 medium in 500 ml pressure resistant bottles were inoculated with centrifuged cells from the first preculture to an OD₆₀₀ₙₘ of 0.03 and flushed with a premixed gas with 67% H₂, 33% CO₂ to an overpressure of 0.8 bar. These growing cultures were incubated at 30° C. and 100 rpm in an open water bath shaker for 22 h to an OD₆₀₀ₙₘ 0.24.

For the main culture, as many centrifuged cells from the second preculture of C. neopropionicum as necessary for an OD₆₀₀ₙₘ of 0.2 were added to 200 mL of spent medium from the main culture of C. autoethanogenum. The cultivation was carried out in a 500 mL pressure-resistant glass bottle at 30° C., 100 rpm and an overpressure of 0.8 bar of a premixed gas with 67% H₂, 33% CO₂ in an open water bath shaker for 114 h. The pH was held at 6.8 by automatic addition of NaOH solution (100 g/L).

During cultivation several 5 mL samples were taken to determinate OD₆₀₀ₙₘ, pH und product formation. The determination of the product concentrations was performed by semi-quantitative ¹H-NMR spectroscopy. As an internal quantification standard sodium trimethylsilylpropionate (T(M)SP) was used.

During the cultivation the concentration of propionate increased from 0.03 g/L to 2.65 g/L, for propanol from 0.006 to 0.07 g/L, for butyrate from 0.003 to 0.16 g/L and for lactate from 0 g/L to 0.035 g/L. The concentration of ethanol decreased from 3.3 g/L to 0.15 g/L during this time.

### REFERENCES

Baffert, C., et al. J. Am. Chem. Soc. 2011, 133, 2096-2099
Barker H A., 1949, J. Biol. Chem. 180: 1085-1093
Bartsch, R. G., 1961, Archives of Biochemistry and biophysics, 92: 122-132
Bornstein B. T., et al., 1948 J Bad, 55:223
Bornstein B. T., et al., 1948 J Biol Chem, 172: 659
Brioukhanov, 2007, Applied Biochemistry and Microbiology, 43 (6): 567-582
Chowdhury N. P., 2014, J. Biol. Chem, 289(8):5145-57
Cotter, J. L (2009) Enzyme and Microbial Technology 44 281-288,
Ding H. et al, 2010, Bioresour Technol, 101(24):9550-9
Drake et al., 2004. Strict and Facultative Anaerobes: Medical and Environmental Aspects. pp. 251-281, Horizon Scientific Press, United Kingdom
Drake & Kusel, 2005 Acetogenic clostridia. In: Durre, P. (ed.), Handbook on Clostridia, pp. 719-746. CRC Press, Boca Raton, Fla.
Drake et al., 2006, Acetogenic prokaryotes. In: Balows A, Truper H G, Dworkin M, Harder W and
Gerhardt, P et al (ed) American Society for Microbiology, Washington, D.C. p. 248-277
Fuchs G., Schlegel H.-G. (2007) AIIgemeine Mikrobiologie, Georg Thieme Verlag, Stuttgart.
Galaction, A.-I. et al., (2012) Chemical Engineering & Technology, 35(9), 1657-1663
Henstra A. M., 2007 Current Opinion in Biotechnology, 18:200-206
Hillmer P., 1972; FEBS Letters; 21(3):351-354
Imlay 2006, Molecular Microbiology, 59(4); 1073-1082
Kandasamy V., 2013, Appl Microbial Biotechnol: 97:1191-1200)
Keshav, Amit et al., Desalination (2009), 244(1-3), 12-23
Koch, A L. 1994. “Growth Measurement IN: Methods for General and Molecular Bacteriology Köpke Michael 2009, Dissertation zur Erlangung des Doktorgrades Dr. rer. nat. der Fakultät für Naturwissenschaften der Universität Ulm
Lan, E. I., Energy Environ. Sci., 6:2672
Li, F., 2008, Journal of Bacteriology, 190 (3): 843-850
Lurz R., 1979; Arch Microbiol; 120: 255-262
Madan, V. K., 1972, Eur. J. Biochem., 32; 51-56
Najafpour. G., 2006 Enzyme and Microbial Technology 38 (2006) 223-228
Perez, J. M. et al., Biotechnology and Bioengineering, 2012, Vol. xxx, No. xxx
Sakai et al., 2004 Biotechnol. Let., Vol. 29, p. 1607-1612
Schleifer K H (eds). The Prokaryotes, 3rd edn. Springer: New York, pp 354-420.
Shuler M L, Kargi F. 1992. Bioprocess Engineering. Prentice Hall, Englewood Cliffs, N.J.
Sliwkowski M. X., 1984, Analytical Biochemistry, 141:344-347
Smith L. T., 1976, Analytical biochemistry, 95:2-7
Smith L. T., 1980, Archives of biochemistry and biophysics, 203 (2): 663-675
Stadtman E. R., 1950, Fed. Proc., 9, 233
Stadtman E. R., 1953, J Biol Chem; 202(2):873-90
Steinbusch, 2011, Energy Environ. Sci., 4, 216-224
Van Eerten-Jansen, M. C. A. A, 2013, ACS Sustainable Chemistry & Engineering 1 (5), 513-518
Wang S, 2010, Journal of Bacteriology, 192 (19): 5115-5123
Winzer K. et al., 1997 Micrbiology 143:3279-3286
Wood, 1991 Life with CO or CO2 and H2 as a source of carbon and energy. FASEB J. 5:156-163
Younesi H. et al. Biochemical Engineering Journal 27 (2005) 110-119
Zhang Y, 2013, Bioprocess Biosyst Eng; 36(12):1897-1904
U.S. 2007/0275447, U.S. 2008/0057554, WO 98/00558, WO 00/68407

1. A method of producing propanol and/or propionic acid from a carbon source in aerobic conditions, the method comprising:
(a) step of producing ethanol and/or acetate from the carbon source in aerobic conditions, comprising
(i) contacting a reaction mixture comprising
a first acetogenic microorganism in an exponential growth phase;
free oxygen; and
a second acetogenic microorganism in a stationary phase
wherein the first and second acetogenic microorganism is capable of converting the carbon source to the acetate and/or ethanol; and
(b) step of contacting the acetate and/or ethanol from step (a) with a third microorganism capable of converting the acetate and/or ethanol to propanol and/or propionic acid.

2. The method according to claim 1, wherein the third microorganism capable of converting the acetate and/or ethanol to propanol and/or propionic acid is a propionogen.

3. The method according to claim 2, wherein the propionogen uses the actate-acrylate pathway for the production of propanol and/or propionic acid.

4. The method according to claim 2, wherein the propionogen uses methylmalonyl-succinate pathway for the production of propanol and/or propionic acid.

5. The method according to claim 2, wherein the C3-producing microorganism is selected from the group consisting of Clostridium neopropionicum, Clostridium propionicum, Pelobacter propionicus, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosyntrophicum, Pelotomaculum thermopropionicum, and Pelotomaculum schinkii.

6. The method according to claim 1, wherein the third microorganism is a genetically modified organism comprising increased expression relative to the wild type cell of propionate CoA-transferase (E₁), lactoyl-CoA dehydratase (E₂) and acryloyl-CoA reductase ((E₃).

7. The method according to claim 1, wherein the first and second microorganism is selected from the group consisting of Clostridium autothenogenum DSMZ 19630, Clostridium ragsdahlei ATCC no. BAA-622, Clostridium autoethanogenum, Moorella sp HUC22-1, Moorella thermoaceticum, Moorella thermoautotrophica, Rumicoccus productus, Acetoanaerobum, Oxobacter pfennigii, Methanosarcina barkeri, Methanosarcina acetivorans, Carboxydothermus, Desulfotomaculum kutznetsovii, Pyrococcus, Peptostreptococcus, Butyribacterium methylotrophicum ATCC 33266, Clostridium formicoaceticum, Clostridium butyricum, Lactobacillus delbrukii, Propionibacterium acidoproprionici, Proprionispera arboris, Anaerobierspirillum succiniproducens, Bacterioides amylophilus, Becterioides ruminicola, Thermoanaerobacter kivui, Acetobacterium woodii, Acetoanaerobium notera, Clostridium aceticum, Butyribacterlum methylotrophicum, Moorella thermoacetica, Eubacterium limosum, Peptostreptococcus productus, Clostridium Ijungdahlii, Clostridium ATCC 29797 and Clostridium carboxidivorans.

8. The method according to claim 1, wherein the first acetogenic microorganism in the exponential growth phase has a growth rate of 0.01 to 2 h⁻¹.

9. The method according to claim 1, wherein the first acetogenic microorganism in the exponential growth phase has an OD₆₀₀ of 0.01 to 2.

10. The method according to claim 1, wherein the aerobic conditions is a result of oxygen being at a concentration of 0.000005-1% volume in the gas phase.

11. The method according to claim 1, wherein the third microorganism is Clostridium neopropionicum.

12. The method according to claim 1, wherein the first and second microorganism is Clostridium Ijungdahlii and the third microorganism is Clostridium neopropionicum.

13. The method according to claim 1, wherein the first and second microorganism is Clostridium autoethanogenum and the third microorganism is Clostridium neopropionicum.

14. The mixture according to claim 1, wherein the carbon source comprises CO.

15. The method according to claim 1, wherein steps (a) and (b) are carried out in a single fermenter.
